(19) United States
(12) Patent Application Publication
(10) Pub. No.: US 2025/0121015 A1
(43) Pub. Date: Apr. 17, 2025

(54) USE OF LACTOBACILLUS PLANTARUM STRAIN, ITS METABOLITE, OR COMPOSITION CONTAINING THE SAME STRAIN FOR REPAIRING OR PREVENTING RETINAL DAMAGE

(71) Applicant: Glac Biotech Co., Ltd., Tainan (TW)

(72) Inventors: Hsieh-Hsun Ho, Tainan (TW); Yu-Chieh Hsu, Tainan (TW); Shin-Yu Tsai, Tainan (TW); Yi-Wei Kuo, Tainan (TW); Min-Rou Sung, Tainan (TW); Ko-Chiang Hsia, Tainan (TW); Jui-Fen Chen, Tainan (TW)

(21) Appl. No.: 18/444,872

(22) Filed: Feb. 19, 2024

(30) Foreign Application Priority Data
Oct. 12, 2023 (TW) 112138931

(51) Int. Cl. A61K 35/747; A61K 35/00; A61K 35/745; A61P 27/02; C12N 1/20; C12R 1/01; C12R 1/225; C12R 1/23; C12R 1/25

(52) U.S. Cl. CPC A61K 35/747; A61K 35/745; A61P 27/02; C12N 1/205; A61K 2035/115; C12R 2001/01; C12R 2001/225; C12R 2001/23; C12R 2001/25

(57) ABSTRACT

The present invention discloses a method for repairing or preventing retinal damage, which includes: administering a composition comprising a Lactobacillus plantarum PL-02 strain or a metabolite thereof to a subject in need thereof; wherein the PL-02 strain is deposited at the China General Microbiological Culture Collection Center with a deposition number CGMCC 20485.

### CROSS REFERENCE

This non-provisional application claims priority of Taiwan Invention Patent Application No. 112138931, filed on Oct. 12, 2023, the contents thereof are incorporated by reference herein.

### FIELD OF THE INVENTION

The present invention is directed to the use of a Lactobacillus plantarum strain and its metabolite, and more particularly to the use of a Lactobacillus plantarum strain, its metabolite, and a composition containing the same for repairing or preventing retinal damage.

### BACKGROUND OF THE INVENTION

Sunlight, LED lamps, and monitors of 3C digital products are the common source of blue light. Blue light has a wavelength from 380 nm to 500 nm and high energy, and belongs to “high-energy visible light (HEV light)”. Blue light is harmful for eyes, and therefore also called as “harmful blue light”. Study shows that blue light can lead to visual fatigue, premature eye aging, and age-related macular degeneration, and blue light can raise cellular oxidative stress with long term exposure to blue light, leading to the death of retinal ganglion cells.

Macular degeneration is also called as “age-related macular degeneration”, which is the third top cause of blindness globally. Macular degeneration is mainly caused by retina aging, and leads to visual deterioration, and possibly to vision loss when the disease becomes serious. Oxidative damage and macular pigment loss caused by light both can lead to macular degeneration. In the ocular photosensitivity test, the participant is more sensitive to short-wavelength light than other light. Short-wavelength light has higher energy, and therefore it is more harmful to eyes. Study indicates that macular pigment has the lowest sensitivity to light with a wavelength of 460 nm, and is able to absorb light for lowering eye discomfort when the light enters the ocular photosensitive layer. With technological advancement, the longer modern people use digital products, the longer they watch monitors. Although there are many products and nutritional supplements for protection from blue light available, they can't effectively block the damage caused by blue light.

Probiotics are living microorganisms and beneficial to host's health. The common bacterial genera are, for example, Lactobacillus spp. and Bifidobacterium spp., and can improve gut microbiota and dysbacteriosis, modulate gut discomfort, improve constipation, protect liver and kidney, protect against aging, and modulate immunity.

Currently, there are attempts to use lactic acid bacteria for improving the eye-related symptoms. For example, Lactobacillus paracasei KW3110 strain can effectively improve visual fatigue caused by blue light and the related disease. However, the probiotic bioactivity is strain-specific, and different bacterial strains belonging to the same species do not have the same activity. That is, bacterial strains belonging to the same species may have different characteristics and effects.

Accordingly, there is a need to develop a composition for improving the eye-related symptoms by discovering the novel activity of lactic acid bacterium strain.

### SUMMARY OF THE INVENTION

Herein, it is found that Lactobacillus plantarum PL-02 strain or its metabolite can reduce damage on human retinal pigment epithelial cells caused by blue-light irradiation. Herein, it is further found that mixing a Lactobacillus plantarum PL-02 strain and other bacterial strains at a specific ratio can synergistically repair damage on human retinal pigment epithelial cells caused by blue-light irradiation. Accordingly, the Lactobacillus plantarum PL-02 strain, its metabolite, and the composition containing the same strain can be used for improving eye discomfort and vision clarity so as to bring out the eye-protection function.

Therefore, the present invention provides a method for repairing or preventing retinal damage, which includes: administering a composition comprising a Lactobacillus plantarum PL-02 strain or a metabolite thereof to a subject in need thereof; wherein the PL-02 strain is deposited at the China General Microbiological Culture Collection Center with a deposition number CGMCC 20485.

Preferably, the retinal damage is retinal damage caused by blue-light irradiation.

Preferably, the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

Preferably, while the composition is a medicine or a nutritional supplement, the composition is administered via topical ocular administration, intraocular administration, or oral administration to the subject; while the composition is a food, the composition is administered via oral administration to the subject.

Preferably, the composition is administered for repairing damage on a human retinal pigment epithelial cell caused by blue-light irradiation.

The present invention additionally provides a method for repairing or preventing retinal damage, which includes: administering a composition to a subject in need thereof; wherein the composition includes: a Lactobacillus plantarum PL-02 strain, a Lactobacillus salivarius subsp. salicinius AP-32 strain, a Lactobacillus plantarum LPL28 strain, a Bifidobacterium longum subsp. infantis BLI-02 strain, and a Lactobacillus acidophilus TYCA06 strain; wherein the PL-02 strain is deposited at the China General Microbiological Culture Collection Center with a deposition number CGMCC 20485; wherein the AP-32 strain is deposited at the China Center for Type Culture Collection with a deposition number CCTCC M2011127; wherein the LPL28 strain is deposited at the China General Microbiological Culture Collection Center with a deposition number CGMCC 17954; wherein the BLI-02 strain is deposited at the China General Microbiological Culture Collection Center with a deposition number CGMCC 15212; wherein the TYCA06 strain is deposited at the China General Microbiological Culture Collection Center with a deposition number CGMCC 15210; wherein based on total bacterial count of the composition, the PL-02 strain is present in an amount from 50 CFU % to 70 CFU %, the AP-32 strain is present in an amount from 7.5 CFU % to 12.5 CFU %, the LPL28 strain is present in an amount from 7.5 CFU % to 12.5 CFU %, the BLI-02 strain is present in an amount from 7.5 CFU % to 12.5 CFU %, and the TYCA06 strain is present in an amount from 7.5 CFU % to 12.5 CFU %.

Preferably, the retinal damage is retinal damage caused by blue-light irradiation.

Preferably, the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

Preferably, while the composition is a medicine or a nutritional supplement, the composition is administered via topical ocular administration, intraocular administration, or oral administration to the subject; while the composition is a food, the composition is administered via oral administration to the subject.

Preferably, the composition is administered for repairing damage on a human retinal pigment epithelial cell caused by blue-light irradiation.

The present invention additionally provides a method for repairing or preventing retinal damage, which includes: administering a composition to a subject in need thereof; wherein the composition includes: a Lactobacillus plantarum PL-02 strain, a Lactobacillus salivarius subsp. salicinius AP-32 strain, a Lactobacillus plantarum LPL28 strain, a Bifidobacterium longum subsp. infantis BLI-02 strain, and a Lactobacillus acidophilus TYCA06 strain; wherein the PL-02 strain is deposited at the China General Microbiological Culture Collection Center with a deposition number CGMCC 20485; wherein the AP-32 strain is deposited at the China Center for Type Culture Collection with a deposition number CCTCC M2011127; wherein the LPL28 strain is deposited at the China General Microbiological Culture Collection Center with a deposition number CGMCC 17954; wherein the BLI-02 strain is deposited at the China General Microbiological Culture Collection Center with a deposition number CGMCC 15212; wherein the TYCA06 strain is deposited at the China General Microbiological Culture Collection Center with a deposition number CGMCC 15210; wherein a colony forming unit ratio of the PL-02 strain to the AP-32 strain to the LPL28 strain to the BLI-02 strain to the TYCA06 strain is (15 to 35): 3.75:3.75:3.75:3.75.

Preferably, the retinal damage is retinal damage caused by blue-light irradiation.

Preferably, the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

Preferably, while the composition is a medicine or a nutritional supplement, the composition is administered via topical ocular administration, intraocular administration, or oral administration to the subject; while the composition is a food, the composition is administered via oral administration to the subject.

Preferably, the composition is administered for repairing damage on a human retinal pigment epithelial cell caused by blue-light irradiation.

### BRIEF DESCRIPTION OF THE DRAWINGS

FIG. 1 is a bar chart showing the comparison of damage repairing extent for different bacterial strains on human retinal pigment epithelial ARPE-19 cells caused by blue-light irradiation; wherein as compared with the control group, ***: P<0.001; as compared with the blue-light irradiation group, ###: P<0.001, #: P<0.05; and

FIG. 2 is a bar chart showing the comparison of damage repairing extent for different bacterial compositions on human retinal pigment epithelial ARPE-19 cells caused by blue-light irradiation; wherein as compared with the control group, ***: P<0.001; as compared with the blue-light irradiation group, ###: P<0.001, ##: P<0.01, #: P<0.05.

### DETAILED DESCRIPTION OF THE INVENTION

The detailed description and preferred embodiments of the invention will be set forth in the following content, and provided for people skilled in the art to understand the characteristics of the invention.

All bacterial strain described herein are deposited at the China General Microbiological Culture Collection Center in No. 3, No. 1 Courtyard, Beichen West Road, Chaoyang District, Beijing City, China, or deposited at the China Center for Type Culture Collection in Wuhan University, Wuhan City, China. The deposition information is listed in Table 1 below.

TABLE 1
Deposition information

| Strain | Deposition number | Deposition date |
|---|---|---|
| Lactobacillus plantarum PL-02 strain | CGMCC 20485 | Aug. 6, 2020 |
| Lactobacillus salivarius subsp. salicinius AP-32 strain | CCTCC M2011127 | Apr. 10, 2011 |
| Lactobacillus plantarum LPL28 strain | CGMCC 17954 | Jun. 18, 2019 |
| Bifidobacterium longum subsp. infantis BLI-02 strain | CGMCC 15212 | Jan. 15, 2018 |
| Lactobacillus acidophilus TYCA06 strain | CGMCC 15210 | Jan. 15, 2018 |

The 16S ribosomal RNA (rRNA) sequencing and the API bacterial identification system are used to identify morphological properties of all bacterial strain described herein. The morphological properties and common properties are listed in Table 2 below.

TABLE 2
Morphological and common properties

| Strain | Morphological properties |
|---|---|
| Lactobacillus plantarum | 1. They are facultative anaerobic bacteria and can survive in a low or |
| PL-02 strain | normal oxygen concentration. The suitable temperature for survival is |
| | from 10° C. to 45° C., and the most suitable is from 30° C. to 35° C. However, |
| | they can still survive in a temperature close to the freezing point. The |
| | suitable pH value for survival is from pH 3.5 to pH 4.2, and the salt |
| | tolerance for survival is from 13% to 15%. |
| | 2. Each body is mainly in the form of a linear rod, and two ends thereof |
| | are in the form of ellipses. The width is usually from 0.9 mm to 1.2 mm, |
| | and the length is usually from 3.0 mm to 8.0 mm. They usually appear |
| | alone, appear in pairs, or arrange in short chains. |
| Lactobacillus salivarius | 1. They are gram-positive and non-sporogenous bacteria without catalase |
| subsp. salicinius | and oxidase and without mobility. They can survive in an aerobic |
| AP-32 strain | environment or anaerobic environment. The most suitable temperature for |
| | survival is 37 ± 1° C.. They are facultative heterofermentative bacteria and |
| | can't produce any gas during glucose metabolism. |
| | 2. Each colony formed on a MRS agar medium is in the form of a white |
| | solid circle. Each body is in the shape of a short rod, and the tail thereof is |
| | in the form of a circle. They usually appear alone. |
| Lactobacillus plantarum | 1. They are gram-positive and non-sporogenous bacteria without catalase |
| LPL28 strain | and oxidase and without mobility. They can survive in an aerobic |
| | environment or anaerobic environment, and the most suitable temperature |
| | for survival is 37 ± 1° C.. They are facultative heterofermentative bacteria |
| | and can't produce any gas during glucose metabolism. |
| | 2. Each colony formed on a MRS agar medium is in the form of a white |
| | solid circle. Each body is in the shape of a short rod, and the tails thereof |
| | are in the form of squares. They usually appear in pairs or arrange in short |
| | chains. |
| Bifidobacterium longum | 1. They are anaerobes and gram-positive and non-sporogenous bacteria |
| subsp. infantis | without catalase and oxidase and without mobility. They can survive in a |
| BLI-02 strain | strict anaerobic environment, and the most suitable temperature for |
| | survival is 37 ± 1° C.. They are facultative heterofermentative bacteria and |
| | can't produce any gas during glucose metabolism. |
| | 2. Each colony formed on a MRS agar medium is in the form of a white |
| | solid circle. Each body is in the shape of a medium rod or long rod, and two |
| | ends thereof are often in the form of branches, e.g. Y-shaped forms or V- |
| | shaped forms. |
| Lactobacillus acidophilus | 1. They are gram-positive and non-sporogenous bacteria without catalase |
| TYCA06 strain | and oxidase and without mobility. They can survive in an aerobic |
| | environment or anaerobic environment, and the most suitable temperature |
| | for survival is 37 ± 1° C.. They are facultative heterofermentative bacteria |
| | and can't produce any gas during glucose metabolism. |
| | 2. Each colony formed on a MRS agar medium is in a translucent irregular |
| | form. Each body is in the shape of a medium rod, and tails thereof are in the |
| | form of circles. They usually appear in pairs or arrange in short chains. |

The first embodiment of the present invention is made based on the discovery that Lactobacillus plantarum PL-02 strain or its metabolite can repair the damage on human retinal pigment epithelial ARPE-19 cells caused by blue-light irradiation. For that reason, the first embodiment provides a method for repairing or preventing retinal damage, the method including: administering a composition comprising a Lactobacillus plantarum PL-02 strain or a metabolite thereof to a subject in need thereof. In an example, the Lactobacillus plantarum PL-02 strain is an active strain or an inactivated strain. In an example, the retinal damage is retinal damage caused by blue-light irradiation. In an example, the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

In an example, the composition is a medicine or a nutritional supplement, and the composition is administered via topical ocular administration, intraocular administration, or oral administration to the subject so as to arrive at the repairing or preventing purpose. In an example, the composition is a food, and the composition is administered via oral administration to the subject so as to arrive at the repairing or preventing purpose. In an example, the composition is administered for repairing damage on a human retinal pigment epithelial cell caused by blue-light irradiation so as to arrive at the repairing or preventing purpose. In an example, the composition is administered at a total bacterial count of the Lactobacillus plantarum PL-02 strain from 10⁶ to 10¹⁰ CFU/kg of body weight of the subject per day so as to arrive at the repairing or preventing purpose; preferably, the composition is administered at a total bacterial count of the Lactobacillus plantarum PL-02 strain from 8.3×10⁷ to 3.4×10⁸ CFU/kg of body weight of the subject per day.

In an example, the composition is a medicine, a food, or a nutritional supplement, and further comprises: a physiologically acceptable excipient, diluent, or carrier.

On the condition of the food or the nutritional supplement, the physiologically acceptable excipient, diluent, or carrier is a food-acceptable excipient, diluent, or carrier. The example thereof is but not limited to a fluid milk (e.g., a milk or a condensed milk), a fermented milk (e.g., a soured milk), a milk powder, an ice cream, a cheese, a cottage cheese, a soy milk, a fermented soy milk, a vegetable juice, a fruit juice, a sports drink, a jelly, a cookie, an energy bar, a health food, an animal feed, or a dietary supplement.

On the condition of the medicine or the nutritional supplement, the physiologically acceptable excipient, diluent, or carrier is a pharmaceutical-acceptable excipient, diluent, or carrier. The example thereof is but not limited to a solvent, a buffer agent, an emulsifying agent, a suspending agent, a decomposing agent, a disintegrant, a dispersant, a binder, a stabilizing agent, a chelating agent, a gelling agent, a humectant, a lubricant, an absorption delaying agent, or a liposome. The medicine or the nutritional supplement may be in a topically ocularly administered dosage, an intraocularly administered dosage, or an orally administered dosage. The example of an orally administered dosage is but not limited to a powder, a tablet, a troche, a lozenge, a pill, a capsule, a solution, a suspension, an emulsion, a syrup, an elixir, a thick slurry, or a drop; the example of a topically ocularly administered dosage is but not limited to a drop, an emulsion, a gel, an ointment, a cream, a spray, or a suspension; the example of an intraocularly administered dosage is but not limited to a drop, an emulsion, a gel, an ointment, a cream, a spray, or a suspension.

The second embodiment of the present invention is made based on the discovery that mixing Lactobacillus plantarum PL-02 strain and other bacterial strains at a specific ratio can synergistically repair the damage on human retinal pigment epithelial ARPE-19 cells caused by blue-light irradiation. For that reason, the second embodiment provides a method for repairing or preventing retinal damage, which includes: administering a composition to a subject in need thereof; wherein the composition includes: a Lactobacillus plantarum PL-02 strain, a Lactobacillus salivarius subsp. salicinius AP-32 strain, a Lactobacillus plantarum LPL28 strain, a Bifidobacterium longum subsp. infantis BLI-02 strain, and a Lactobacillus acidophilus TYCA06 strain; wherein the following is satisfied: (1) based on total bacterial count of the composition, the Lactobacillus plantarum PL-02 strain is present in an amount from 50 CFU % to 70 CFU %, the Lactobacillus salivarius subsp. salicinius AP-32 strain is present in an amount from 7.5 CFU % to 12.5 CFU %, the Lactobacillus plantarum LPL28 strain is present in an amount from 7.5 CFU % to 12.5 CFU %, the Bifidobacterium longum subsp. infantis BLI-02 strain is present in an amount from 7.5 CFU % to 12.5 CFU %, and the Lactobacillus acidophilus TYCA06 strain is present in an amount from 7.5 CFU % to 12.5 CFU %; or (2) a colony forming unit ratio of the Lactobacillus plantarum PL-02 strain to the Lactobacillus salivarius subsp. salicinius AP-32 strain to the Lactobacillus plantarum LPL28 strain to the Bifidobacterium longum subsp. infantis BLI-02 strain to the Lactobacillus acidophilus TYCA06 strain is (15 to 35): 3.75:3.75:3.75:3.75.

In an example, based on total bacterial count of the composition, the Lactobacillus plantarum PL-02 strain is present in an amount of 50 CFU %, the Lactobacillus salivarius subsp. salicinius AP-32 strain is present in an amount of 12.5 CFU %, the Lactobacillus plantarum LPL28 strain is present in an amount of 12.5 CFU %, the Bifidobacterium longum subsp. infantis BLI-02 strain is present in an amount of 12.5 CFU %, and the Lactobacillus acidophilus TYCA06 strain is present in an amount of 12.5 CFU %.

In an example, the colony forming unit ratio of the Lactobacillus plantarum PL-02 strain to the Lactobacillus salivarius subsp. salicinius AP-32 strain to the Lactobacillus plantarum LPL28 strain to the Bifidobacterium longum subsp. infantis BLI-02 strain to the Lactobacillus acidophilus TYCA06 strain is 15:3.75:3.75:3.75:3.75.

In an example, the Lactobacillus plantarum PL-02 strain, the Lactobacillus salivarius subsp. salicinius AP-32 strain, the Lactobacillus plantarum LPL28 strain, the Bifidobacterium longum subsp. infantis BLI-02 strain, and the Lactobacillus acidophilus TYCA06 strain are individually an active strain or an inactivated strain. In an example, the retinal damage is retinal damage caused by blue-light irradiation. In an example, the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

In an example, the composition is a medicine or a nutritional supplement, and the composition is administered via topical ocular administration, intraocular administration, or oral administration to the subject so as to arrive at the repairing or preventing purpose. In an example, the composition is a food, and the composition is administered via oral administration to the subject so as to arrive at the repairing or preventing purpose. In an example, the composition is administered for repairing damage on a human retinal pigment epithelial cell caused by blue-light irradiation so as to arrive at the repairing or preventing purpose. In an example, the composition is administered at a total bacterial count of the Lactobacillus plantarum PL-02 strain, the Lactobacillus salivarius subsp. salicinius AP-32 strain, the Lactobacillus plantarum LPL28 strain, the Bifidobacterium longum subsp. infantis BLI-02 strain, and the Lactobacillus acidophilus TYCA06 strain from 10⁶ to 10¹⁰ CFU/kg of body weight of the subject per day so as to arrive at the repairing or preventing purpose; preferably, the composition is administered at a total bacterial count of the Lactobacillus plantarum PL-02 strain, the Lactobacillus salivarius subsp. salicinius AP-32 strain, the Lactobacillus plantarum LPL28 strain, the Bifidobacterium longum subsp. infantis BLI-02 strain, and the Lactobacillus acidophilus TYCA06 strain from 8.3×10⁷ to 3.4×10⁸ CFU/kg of body weight of the subject per day.

In an example, the composition is a medicine, a food, or a nutritional supplement, and further comprises: a physiologically acceptable excipient, diluent, or carrier.

On the condition of the food or the nutritional supplement, the physiologically acceptable excipient, diluent, or carrier is a food-acceptable excipient, diluent, or carrier. The example thereof is but not limited to a fluid milk (e.g., a milk or a condensed milk), a fermented milk (e.g., a soured milk), a milk powder, an ice cream, a cheese, a cottage cheese, a soy milk, a fermented soy milk, a vegetable juice, a fruit juice, a sports drink, a jelly, a cookie, an energy bar, a health food, an animal feed, or a dietary supplement.

On the condition of the medicine or the nutritional supplement, the physiologically acceptable excipient, diluent, or carrier is a pharmaceutical-acceptable excipient, diluent, or carrier. The example thereof is but not limited to a solvent, a buffer agent, an emulsifying agent, a suspending agent, a decomposing agent, a disintegrant, a dispersant, a binder, a stabilizing agent, a chelating agent, a gelling agent, a humectant, a lubricant, an absorption delaying agent, or a liposome. The medicine or the nutritional supplement may be in a topically ocularly administered dosage, an intraocularly administered dosage, or an orally administered dosage. The example of an orally administered dosage is but not limited to a powder, a tablet, a troche, a lozenge, a pill, a capsule, a solution, a suspension, an emulsion, a syrup, an elixir, a thick slurry, or a drop; the example of a topically ocularly administered dosage is but not limited to a drop, an emulsion, a gel, an ointment, a cream, a spray, or a suspension; the example of an intraocularly administered dosage is but not limited to a drop, an emulsion, a gel, an ointment, a cream, a spray, or a suspension.

The following examples are offered for further illustrating the invention.

### Example 1: Cultivation of Bacterial Strain

The bacterial strains used herein include: Lactobacillus plantarum PL-02 strain, Lactobacillus salivarius subsp. salicinius AP-32 strain, Lactobacillus plantarum LPL28 strain, Bifidobacterium longum subsp. infantis BLI-02 strain, Lactobacillus acidophilus TYCA06 strain, and Lactobacillus paracasei KW3110 strain.

A bacterial strain is seeded into an MRS broth medium containing 0.05% cysteine, and then cultivated in an incubator at a temperature of 37° C. with a CO₂ concentration of 5% for 24 hours to activate the strain. After which, the bacterial strain is pelleted and suspended with a fresh MRS broth medium to adjust its concentration to 109 CFU/mL.

### Example 2: In Vitro Test for Reducing Cell Damage Caused by Blue Light for Single Bacterial Strain

MTT assay is used to analyze the cell survival rate. In order to model the oral administration for probiotic, each single bacterial strain is incubated with human colorectal adenocarcinoma Caco-2 cells for 6 hours, and the bacterial counts of all single bacterial strains are the same. After which, the thus-obtained culture is centrifuged at a rate of 4,000 rpm for 10 minutes so as to obtain a supernatant. The supernatant is incubated with human retinal pigment epithelial ARPE-19 cells overnight, and the cells are irradiated with blue light during incubation. After removing the culture medium and washing the cells with phosphate buffered saline (PBS), MTT assay is performed to detect the cell survival rate. Specifically, MTT (3-(4,5-dimethylthiazol-2-yl)-2,5-diphenyl tetrazolium bromide) is added into each well, and then reacted with cells in an incubator at a temperature of 37° C. with a CO₂ concentration of 5% for from 1 hour to 4 hours. After removing MTT, an enzyme-linked immunosorbent assay (ELISA) reader is used to detect the absorbance at a wavelength of 570 nm. Finally, the average absorbance in the control group divides the average absorbance in the experimental group to obtain the cell survival rate in the experiment group.

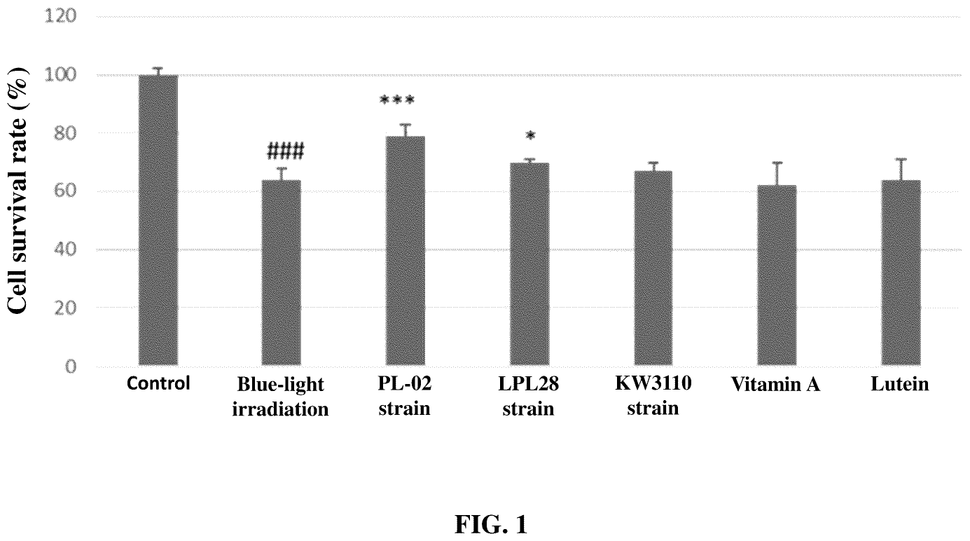

As shown in FIG. 1, the survival rate of ARPE-19 cells decreases to 64% after blue-light irradiation. As compared with effects on the survival rate after blue-light irradiation for various single bacterial strains, Lactobacillus plantarum PL-02 strain can increase the survival rate to 79%, and Lactobacillus plantarum LPL28 strain can increase the survival rate to 70%, which implies that repairing the damage on human retinal pigment epithelial cells caused by blue light for Lactobacillus plantarum is strain specific. The common components for eye protection, vitamin A and lutein, can increase the survival rate to 62% and 64%, respectively, and the commercial bacterial strain for eye protection, Lactobacillus paracasei KW3110 strain, can increase the survival rate to 67%.

### Example 3: In Vitro Test for Reducing Cell Damage Caused by Blue Light for Bacterial Strain Mixture

Amounts of different bacterial strains in all mixture groups are listed in Table 3.

TABLE 3
Bacterial strain amount

| | | Lactobacillus | | Bifidobacterium | |
|---|---|---|---|---|---|
| | Lactobacillus | salivarius subsp. | Lactobacillus | longum subsp. | Lactobacillus |
| | plantarum | salicinius | plantarum | infantis | acidophilus |
| | PL-02 strain | AP-32 strain | LPL28 strain | BLI-02 strain | TYCA06 strain |
| mixture | 80 CFU % | 5 CFU % | 5 CFU % | 5 CFU % | 5 CFU % |
| group 1 | | | | | |
| mixture | 70 CUF % | 7.5 CFU % | 7.5 CFU % | 7.5 CFU % | 7.5 CFU % |
| group 2 | | | | | |
| mixture | 50 CFU % | 12.5 CFU % | 12.5 CFU % | 12.5 CFU % | 12.5 CFU % |
| group 3 | | | | | |
| mixture | 30 CFU % | 17.5 CFU % | 17.5 CFU % | 17.5 CFU % | 17.5 CFU % |
| group 4 | | | | | |
| mixture | 20 CFU % | 20 CFU % | 20 CFU % | 20 CFU % | 20 CFU % |
| group 5 | | | | | |
| mixture | 0 CFU % | 25 CFU % | 25 CFU % | 25 CFU % | 25 CFU % |
| group 6 | | | | | |

MTT assay is used to analyze the cell survival rate. In order to model the oral administration for probiotic, each group is incubated with human colorectal adenocarcinoma Caco-2 cells for 6 hours, and the total bacterial counts of all single bacterial strain groups and all mixture groups are the same. After which, the thus-obtained culture is centrifuged at a rate of 4,000 rpm for 10 minutes so as to obtain a supernatant. The supernatant is incubated with human retinal pigment epithelial ARPE-19 cells overnight, and the cells are irradiated with blue light during incubation. After removing the culture medium and washing the cells with phosphate buffered saline (PBS), MTT assay is performed to detect the cell survival rate. Specifically, MTT (3-(4,5-dimethylthiazol-2-yl)-2,5-diphenyl tetrazolium bromide) is added into each well, and then reacted with cells in an incubator at a temperature of 37° C. with a CO₂ concentration of 5% for from 1 hour to 4 hours. After removing MTT, an enzyme-linked immunosorbent assay (ELISA) reader is used to detect the absorbance at a wavelength of 570 nm. Finally, the average absorbance in the control group divides the average absorbance in the experimental group to obtain the cell survival rate in the experiment group.

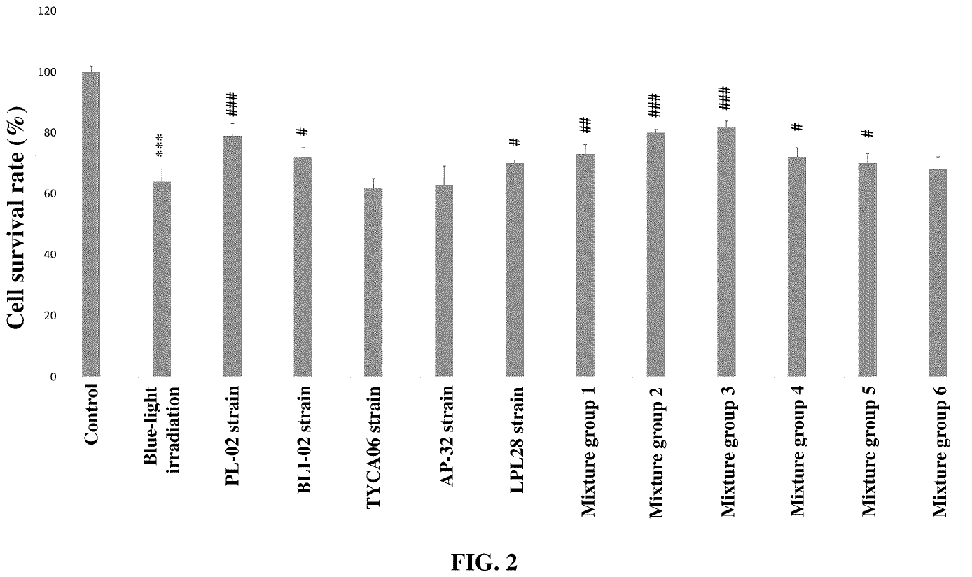

As shown in FIG. 2, the survival rate of ARPE-19 cells decreases to 64% after blue-light irradiation. As compared with effects on the survival rate after blue-light irradiation for various single bacterial strains, Lactobacillus plantarum PL-02 strain can increase the survival rate to 79%, Bifidobacterium longum subsp. infantis BLI-02 strain can increase the survival rate to 72%, Lactobacillus acidophilus TYCA06 strain can decrease the survival rate to 62%, Lactobacillus salivarius subsp. salicinius AP-32 strain can decrease the survival rate to 63%, and Lactobacillus plantarum LPL28 strain can increase the survival rate to 70%. As compared with effects on the survival rate after blue-light irradiation for various mixture groups, the mixture groups 1-6 can increase the survival rates to 73%, 80%, 82%, 72%, 70%, and 68%, respectively. Especially, the survival rates for the mixture groups 2 and 3 are higher than that of any single bacterial strain group, which implies the bacterial count ratios defined in the mixture groups 2 and 3 have a synergistic increase of the cell survival rate after blue-light irradiation.

Herein, the cell repair rate after blue-light irradiation for the experimental group is defined as below:

$$Repair rate = \frac{Cell survival rate after blue light irradiation for experimental group - Cell survival rate after mere blue light irradiation}{Cell survival rate without blue light irradiation - Cell survival rate after mere blue light irradiation}$$

The repair rates for all single bacterial strain groups and all mixture groups are listed in Table 4.

TABLE 4
Repair rate

| | Repair rate (%) |
|---|---|
| PL-02 strain | 41.67 |
| BLI-02 strain | 22.22 |
| TYCA06 strain | −5.56 |
| AP-32 strain | −2.78 |
| LPL28 strain | 16.67 |
| mixture group 1 | 25.00 |
| mixture group 2 | 44.44 |
| mixture group 3 | 50.00 |
| mixture group 4 | 22.22 |
| mixture group 5 | 16.67 |
| mixture group 6 | 11.11 |

As compared with effects on the repair rate after blue-light irradiation for various mixture groups, the repair rates for the mixture groups 2 and 3 are higher than that of any single bacterial strain group, which implies the bacterial count ratios defined in the mixture groups 2 and 3 have synergistic improvement on the cell repair after blue-light irradiation.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for repairing or preventing retinal damage, comprising:
administering a composition comprising a Lactobacillus plantarum PL-02 strain or a metabolite thereof to a subject in need thereof; wherein the PL-02 strain is deposited at the China General Microbiological Culture Collection Center with a deposition number CGMCC 20485.

2. The method as claimed in claim 1, wherein the retinal damage is retinal damage caused by blue-light irradiation.

3. The method as claimed in claim 1, wherein the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

4. The method as claimed in claim 1, wherein the composition is a medicine or a nutritional supplement, and the composition is administered via topical ocular administration, intraocular administration, or oral administration to the subject.

5. The method as claimed in claim 1, wherein the composition is a food, and the composition is administered via oral administration to the subject.

6. The method as claimed in claim 1, wherein the composition is administered for repairing damage on a human retinal pigment epithelial cell caused by blue-light irradiation.

7. A method for repairing or preventing retinal damage, comprising:
administering a composition to a subject in need thereof, wherein the composition includes: a Lactobacillus plantarum PL-02 strain, a Lactobacillus salivarius subsp. salicinius AP-32 strain, a Lactobacillus plantarum LPL28 strain, a Bifidobacterium longum subsp. infantis BLI-02 strain, and a Lactobacillus acidophilus TYCA06 strain; wherein the PL-02 strain is deposited at the China General Microbiological Culture Collection Center with a deposition number CGMCC 20485; wherein the AP-32 strain is deposited at the China Center for Type Culture Collection with a deposition number CCTCC M2011127; wherein the LPL28 strain is deposited at the China General Microbiological Culture Collection Center with a deposition number CGMCC 17954; wherein the BLI-02 strain is deposited at the China General Microbiological Culture Collection Center with a deposition number CGMCC 15212; wherein the TYCA06 strain is deposited at the China General Microbiological Culture Collection Center with a deposition number CGMCC 15210; wherein based on total bacterial count of the composition, the PL-02 strain is present in an amount from 50 CFU % to 70 CFU %, the AP-32 strain is present in an amount from 7.5 CFU % to 12.5 CFU %, the LPL28 strain is present in an amount from 7.5 CFU % to 12.5 CFU %, the BLI-02 strain is present in an amount from 7.5 CFU % to 12.5 CFU %, and the TYCA06 strain is present in an amount from 7.5 CFU % to 12.5 CFU %.

8. The method as claimed in claim 7, wherein based on total bacterial count of the composition, the Lactobacillus plantarum PL-02 strain is present in an amount of 50 CFU %, the Lactobacillus salivarius subsp. salicinius AP-32 strain is present in an amount of 12.5 CFU %, the Lactobacillus plantarum LPL28 strain is present in an amount of 12.5 CFU %, the Bifidobacterium longum subsp. infantis BLI-02 strain is present in an amount of 12.5 CFU %, and the Lactobacillus acidophilus TYCA06 strain is present in an amount of 12.5 CFU %.

9. The method as claimed in claim 7, wherein the retinal damage is retinal damage caused by blue-light irradiation.

10. The method as claimed in claim 7, wherein the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

11. The method as claimed in claim 7, wherein the composition is a medicine or a nutritional supplement, and the composition is administered via topical ocular administration, intraocular administration, or oral administration to the subject.

12. The method as claimed in claim 7, wherein the composition is a food, and the composition is administered via oral administration to the subject.

13. The method as claimed in claim 7, wherein the composition is administered for repairing damage on a human retinal pigment epithelial cell caused by blue-light irradiation.

14. A method for repairing or preventing retinal damage, comprising:
administering a composition to a subject in need thereof; wherein the composition includes: a Lactobacillus plantarum PL-02 strain, a Lactobacillus salivarius subsp. salicinius AP-32 strain, a Lactobacillus plantarum LPL28 strain, a Bifidobacterium longum subsp. infantis BLI-02 strain, and a Lactobacillus acidophilus TYCA06 strain; wherein the PL-02 strain is deposited at the China General Microbiological Culture Collection Center with a deposition number CGMCC 20485; wherein the AP-32 strain is deposited at the China Center for Type Culture Collection with a deposition number CCTCC M2011127; wherein the LPL28 strain is deposited at the China General Microbiological Culture Collection Center with a deposition number CGMCC 17954; wherein the BLI-02 strain is deposited at the China General Microbiological Culture Collection Center with a deposition number CGMCC 15212; wherein the TYCA06 strain is deposited at the China General Microbiological Culture Collection Center with a deposition number CGMCC 15210; wherein a colony forming unit ratio of the PL-02 strain to the AP-32 strain to the LPL28 strain to the BLI-02 strain to the TYCA06 strain is (15 to 35): 3.75:3.75:3.75:3.75.

15. The method as claimed in claim 14, wherein the colony forming unit ratio of the Lactobacillus plantarum PL-02 strain to the Lactobacillus salivarius subsp. salicinius AP-32 strain to the Lactobacillus plantarum LPL28 strain to the Bifidobacterium longum subsp. infantis BLI-02 strain to the Lactobacillus acidophilus TYCA06 strain is (15 to 35): 3.75:3.75:3.75:3.75.

16. The method as claimed in claim 14, wherein the retinal damage is retinal damage caused by blue-light irradiation.

17. The method as claimed in claim 14, wherein the retinal damage includes: macular degeneration, retinitis pigmentosa, diabetic retinopathy, non-proliferative diabetic retinopathy, proliferative diabetic retinopathy, hypertensive retinopathy, retinopathy of prematurity, radiation retinopathy, or solar retinopathy.

18. The method as claimed in claim 14, wherein the composition is a medicine or a nutritional supplement, and the composition is administered via topical ocular administration, intraocular administration, or oral administration to the subject.

19. The method as claimed in claim 14, wherein the composition is a food, and the composition is administered via oral administration to the subject.

20. The method as claimed in claim 14, wherein the composition is administered for repairing damage on a human retinal pigment epithelial cell caused by blue-light irradiation.
